(19) United States
(12) Patent Application Publication
(10) Pub. No.: US 2008/0260692 A1
(43) Pub. Date: Oct. 23, 2008

(54) Spirulin Composition Rich in Active Principles, Method for Obtaining Same and Use Thereof

(76) Inventor: Jean-Louis Vidalo, Brive-La-Gaillarde (FR)

Correspondence Address: BROWDY AND NEIMARK, P.L.L.C.;624 NINTH STREET, NW, SUITE 300, WASHINGTON, DC, 20001-5303, US

(21) Appl. No.: 11/908,930

(22) PCT Filed: Mar. 16, 2006
(86) PCT No.: PCT/FR2006/000569

§ 371 Date: May 9, 2008

(30) Foreign Application Priority Data
Mar. 16, 2005 (FR) 0502606

(51) Int. Cl. A61K 35/74; C12N 1/12; A61K 8/99

(52) U.S. Cl. 424/93.1; 435/257.1

(57) ABSTRACT

The invention relates to a spirulina combination composed of at least two distinct strain types from the genus Arthrospira, particularly Arthrospira LONAR and Arthrospira PARACAS whose phycocyanin content is between 13 and 30%, a method for obtaining such a combination, a composition containing this combination, and its use as a therapeutic and/or cosmetic agent.

The present invention concerns compositions comprising spirulina of the Arthrospira sp. LONAR and Arthrospira sp. PRACAS type, which can have very high contents of certain compounds, particularly phycocyanin.

Spirulina are filamentous cyanobacteria that belong to the genus Arthrospira or Spirulina.

They are microscopic photosynthetic algae that grow naturally in the alkaline waters of certain lakes in the intertropical zone.

Once used by the Aztecs of ancient Mexico as an energy food, spirulina was rediscovered in the 1960s during periods of drought in sub-Saharan Africa. It was found that, under those famine conditions, the Kanembu, a community on the rim of Lake Chad that had traditionally consumed cakes of dried spirulina (dihé), did not show any symptoms of malnutrition.

Spirulina then became the subject of in-depth research, which revealed that this microorganism had exceptional nutritional properties.

The research was initially aimed at identifying the various species of spirulina and the conditions under which they could be obtained; subsequently, various pilot projects were established in subtropical countries under the auspices of development and malnutrition prevention programs [Fox, R. D., Algoculture: Spirulina, Hope for a Hungry World. Pub. by Edisud, Aix-en-Provence, France, 1986].

One of the advantages of spirulina is that it grows in low- to high-salinity alkaline waters, i.e. in conditions where the growth of other microorganisms is limited. As a result, it is fairly easy to obtain uncontaminated homogeneous cultures in relatively short periods of time.

Another advantage of spirulina is linked to the high yields of the cultures. For example, after 50 days, one can expect to harvest 9 kg of dry matter from one gram of dry spirulina seed under conventional culture conditions, i.e. in a 15-cm deep basin with water at 32° C., a pH of around 9-10 and 16 hours of sunlight. Given that more than half or the dry mass of spirulina is composed of proteins, the protein yield obtained, for a comparable surface area, is 20 times that of a field of soy [Jourdan, J. P., Cultivez votre spiruline].

After harvest and filtration, the spirulina is in the form of a dark green paste, which can be eaten fresh on the spot or dehydrated. The powder obtained has a salty taste. It can be preserved and incorporated into various foods as a dietary supplement.

Spirulina has been subjected to exhaustive toxicological studies, in light of which it is possible to assert that it is perfectly harmless.

Spirulina, mixed with a base food, makes it possible to overcome moderate to acute malnutrition. For example, a child suffering from kwashiorkor (Vitamin B12 deficiency) can be restored to health by being given a spoonful of spirulina a day for one month.

In addition to the importance of spirulina cultures in assisting populations affected by malnutrition, spirulina is enjoying increasing success in industrialized nations among athletes and nutritionists, who find this natural product to be an exceptional source of active ingredients.

In particular, it has significant contents of:
iron, particularly iron that is bioavailable, i.e. directly absorbable,
vitamin B9, or folic acid, which participates in iron binding and muscle anabolism,
branched chain amino acids—BCAAs—leucine, isoleucine, valine—which are particularly sought-after for adding muscle mass,
vitamins B1, B6 and B12, which play an essential role in both the preparation for and recovery from effort,
the liposoluble vitamins D, E, A, and above all provitamin A—beta carotene—which eliminates the risk of overdosage,
metals (in non-toxic proportions): selenium, copper, magnesium, manganese,
enzymes and co-enzymes involved in metabolic reactions, including superoxide dismutase (SOD), which is a powerful antioxidant,
polyunsaturated omega-3 and omega-6 fatty acids, and more particularly linolenic and di-homo-gamma-linolenic acid, which are essential for the body; and
phycocyanin, the primary photosynthetic pigment of spirulina, which also has antioxidant properties.

Of these active ingredients, phycocyanin, SOD and the polyunsaturated fatty acids di-homo-gamma-linolenic and gamma-linolenic acid are the most sought-after.

Phycocyanin is a 332-kd phycobiliprotein that is similar in structure to human biliary pigments, which are known for their anti-inflammatory properties. Studies have pointed out that phycocyanin makes it possible, in animals, to inhibit the formation of B4 leukotrienes, which are highly inflammatory eicosanoid metabolites, as well as the activity of cyclo-oxygenase-2 (COX-2) and lipoxygenase, which are enzymes associated with inflammatory processes [Romay, C., et al., Inflamm. Res., 1998, 47(1):36-41].

Other studies have shown that the phycocyanin extracted from spirulina, injected into the blood stream, stimulates hematopoiesis [Zhang Cheng-Wu, et al., Effects of polysaccharide and phycocyanin from spirulina on peripheral blood and hematopoietic system of bone marrow in mice. Second Asia-Pacific Conf., April, 1994].

Phycocyanin is also used as a food colorant (absorption peak visible at 620 nm) and as a molecular biology reagent for the detection of proteins and more particularly antibodies [Boussiba, S., and Richmond, A. E., C-Phycocyanins as a storage protein in the blue-green algae Spirulina platensis, 1980, Arch. Microbiol. 125: 143-147].

Superoxide dismutase (SOD) is an enzyme that has the power to deactivate the superoxide ions generated by enzymes such as NADPH oxidase or cytochrome oxidase. These very short-lived free radicals are highly toxic for biological molecules, particularly for polyunsaturated fatty acids, in which they produce chain peroxidations. SOD binds the superoxide ions to hydrogen ions to produce less-toxic molecular oxygen and hydrogen peroxide. Thus, SOD plays a detoxifying role, but also a protective role against the oxidation reactions that can occur with infections or tumor formation.

Di-homo-gamma-linolenic and gamma-linolenic acid are polyunsaturated fatty acids of the omega-6 family. These polyunsaturated fatty acids are essential for maintaining membranes in every organ of the human body. Thus, they play an essential part in all of the biological functions of the human body.

Studies of nutritional supplements show that in many respects, spirulina can alleviate the symptoms of many diseases [Belay, A., Ota, Y. et al. Current knowledge on potential health benefits of spirulina, Journal of Applied Phycology, 1993, 5:235-241]. In particular, it can reduce anemia, cholesterol levels and the risk of hypertension, protect the body against free radical attacks, and stimulate the immune system, particularly in the case of viral attacks.

In the context of the use of spirulina for health or athletic purposes, a spirulina, composition is currently considered to be of excellent quality when it comprises a phycocyanin content of around 10% by dry weight, a superoxide dismutase activity of around 30 to 40,000 IU per 100 g of dried spirulina, a gamma-linoleic acid content of around 10%, and a di-homo-gamma-linolenic acid content of about 0.5%.

A spirulina of this nature is obtained, for example using the Arthrospira pacifica strain, in industrial production plants located in the United States [Henrikson, R., Earth Food Spirulina, 1989, Ronore Enterprises Inc.].

The most commonly cultivated spirulina strains, however, are Arthrospira platensis and Arthrospira maxima, which do not male it possible to obtain a spirulina of equivalent quality in an industrial setting.

An example of cultures which do make it possible, using these strains, to obtain a spirulina with improved fatty acid content is described in the patent application WO 9915688.

Morphological differences may appear as a function of the spirulina species cultivated. Thus, the spirulina filaments have a more or less spiraled appearance, reflecting the differences between one species and another in adapting to the culture conditions. As a result, certain strains are reputed to have a more or less typical morphology.

In the recommendations given to spirulina growers, it is advised that only one type of strain, particularly a spiraled strain, be used, in order to better control the various culture stages and more easily detect contaminations. For example, a strain like Arthrospira sp. PARACAS, which is clearly spiraled and has a dark green color under standard culture conditions, is not likely to be suspected of not being a true spirulina. On the other hand, a strain like Arthrospira sp. LONAR, which is paler and has a wavy appearance, is easily confused with algae of the Oscillatoria type, which do not have the same advantages as spirulina. It is also noted that many species of toxic cyanophyceae can grow in the culture media when the spirulina cultures are not properly controlled, which explains a certain mistrust on the part of growers toward species having a less typical appearance.

However, contrary to accepted notions, the inventor discovered that cultivating different strains of spirulina concurrently, particularly strains of the Arthrospira sp. LONAR and PARADAS type, under well defined conditions, makes it possible to significantly improve the quality of the spirulina.

In particular, he discovered that co-culturing spirulina strains makes it possible to obtain compositions whose phycocyanin and superoxide dismutase content is greater than what had been previously described.

These compositions also have a high di-homo-gamma-linoleic and gamma-lineolenic acid content, which gives them a very high quality index.

The compositions obtained can be used in many applications, particularly as dietary supplements or medications, and particularly in order to offset deficiencies in the immune system and support physical effort.

They can also be used as cosmetic products, particularly dermatological products.

Thus, the subject of the present invention is a spirulina combination composed of at least two strain types from the genus Arthrospira, characterized in that the average phycocyanin content of said strains is between 13 and 30%, and preferably between 14 and 25%.

The term spirulina designates a blue-green alga belonging to the phylum Cyanophyta, the class Cyanophycaea, the order Nostocales, the family Oscillatoriacaea, and the genus Spirulina or Arthrospira.

The term spirulina combination designates the presence of several species or subspecies (types) of spirulina in the same culture. In the harvested product, this combination is manifested by the simultaneous presence of cells belonging to different spirulina types, in non-residual quantities.

For the sale of consistency in the description, the percentages and values in the present application are expressed in total dry weight of the combinations or compositions in question.

Preferably, the spirulina combination is composed of two strain types in a weight ratio that is generally 80:20, and preferably 40:60.

A combination according to the invention generally comprises SOD having an activity of between 60,000 and 300,000 IU, and preferably between 160,000 and 250,000 TU per 100 g of said combination.

IUs (International Units) reflect the quantity of enzyme that catalyzes the transformation of 1 μmol of substrate per minute, i.e. in the case of SOD, the neutralization of 1 μmol of superoxide ions. The enzymatic activity of superoxide dismutase can be determined by spectrophotometry, for example using the method described by Kuthan, H., et al., [A spectrophotometric assay for superoxide dismutase activities in crude tissue fractions, 1986, Biochem J. 237(1):175-80].

More preferably, a combination according to the invention is composed of the two strain types Arthrospira sp. LONAR and Arthrospira SP. PARACAS.

The combination of these two strains has proven to be particularly advantageous for obtaining a high content of the desired active ingredients, particularly when these strains are cultivated according to the method described in the present application.

The strains Arthrospira sp. LONAR and Arthrospira sp. PARACAS are strains that initially came from Lonar Lake in India and from old water storage basins in Paracas, Peru.

It is possible to obtain strains of this type, i.e. having the same phenotypical and genetic characteristics, from a number of research institutes worldwide, in particular the Pasteur Institute.

Another subject of the invention consists in compositions having a spirulina combination as defined above, particularly a composition comprising:

10-70% spirulina of the Arthrospira sp. LONAR type;

10-70% spirulina of the Arthrospira sp. PARACAS type;

0-30% spirulina of another type.

Spirulina of another type means any other strain of spirulina that is revealed to be compatible with, and preferably complementary to, the combination formed by Arthrospira sp. LONAR and Arthrospira sp. PARACAS.

The compositions according to the invention are more particularly characterized by a phycocyanin content of between 13 and 30%, and preferably between 14 and 28% of the total dry weight of said composition.

A composition according to the invention is also characterized in that it comprises a di-homo-gamma-linolenic acid content that is generally between 0.05% and 2%, preferably between 0.1 and 1.5%, and more preferably between 0.2 and 0.8% of the total dry weight of said composition. It may also comprise a gamma-linolenic acid content that is generally between 0.5 and 5%, preferably between 1 and 3%, and more preferably between 1.5 and 2.5% of the total dry weight of the composition.

Furthermore, a composition according to the invention may comprise superoxide dismutase (SOD) or any other enzyme having the same activity. The activity measured in 1000 g of dry weight of said composition is preferably between 60,000 and 300,000 IU, and more preferably between 160,000 and 250,000 IU.

The term composition designates both the harvested product of a spirulina combination as defined above and any composition resulting from the transformation of this harvested product into a derivative product comprising at least two types of spirulina and having a phycocyanin content greater than 13%, preferably greater than 14%, and more preferably greater than 15%. The compositions according to the invention can be essentially composed of spirulina, but can also result from the mixture or treatment of the spirulina with other products, such as artificial or non-artificial additives, other active ingredients or adjuvants such as, for example, coating agents.

A composition according to the invention may be solid, liquid, or in the form of a powder, simple or sugar-coated tablets, capsules, granules, chewables, suppositories or syrups.

Preferred compositions according to the invention may comprise, in addition to the above-described combinations, at least one active ingredient from among the following: isoflavone, vitamin C, bamboo exudates and Pygeum africanum extracts.

Isoflavone can influence aging, in that it stimulates the secretion of DHEA, which may be lacking in elderly persons, particularly menopausal women. The spirulina combination according to the invention is complementary to the action of isoflavone, in that the active ingredients contained in the spirulina protect and repair tissues damaged by free radicals.

Vitamin C is practically the only vitamin that is not found in spirulina. Thus, it can be useful to add it to the compositions according to the invention in order to produce compositions that meet all of the body's oligoelement requirements.

Bamboo exudates, which are rich in silicon, are known to limit bone degeneration. Along with the vitamin B9 contained in the spirulina combinations, they make it possible to limit the demineralization phenomenon that leads to osteoporosis.

Another subject of the invention consists in a method for obtaining a composition or a combination of spirulina strains according to the invention.

This method comprises the following steps:

a) a spirulina strain, and preferably two or more, is/are cultivated in the same culture medium; and

b) the spirulina cultivated in a) are harvested.

A preferred method according to the invention is a method that makes it possible not only to co-cultivate strains but to obtain a high content of the desired active ingredients. Such a method comprises the following steps:

i) at least one strain of spirulina, and preferably two or more, is/are cultured in a culture medium whose pH is between 8 and 12, and preferably between 9 and 11; and

ii) physicochemical stress is applied to the culture medium by varying the pH within a range of 1 to 3 points, during a luminosity peak.

Applying physicochemical stress is a step that combines strong illumination with a lowering of the pH. The inventors observed that these conditions impose physiological adaptations on the spirulina, resulting in an increased synthesis of pigment and other antioxidant active ingredients such as SOD.

In their natural environment, the strains Arthrospira sp. LONAR and PARACAS do not occupy the same ecological niche. Moreover, they do not have the same needs, particularly in terms of carbon and nitrogen. Spirulina strains of the PARACAS type are somewhat pelagic (they float between the surface and the bottom), whereas strains of the LONAR type occupy the surface of a body of water, where they tend to form aggregates.

Notwithstanding these differences, the method according to the invention is particularly adapted to the co-culture of the two strain types Arthrospira sp. LONAR and Arthrospira sp. PARACAS, either alone or in a mixture with another strain type.

This method makes it possible to obtain particularly high contents of the desired active ingredients, as indicated above, particularly when it comes to phycocyanin, fatty acid and SOD content.

In order to make the method more efficient, it is recommended that the culture medium be allowed to rest in the dark prior to harvesting the spirulina.

This rest phase is generally carried out without any stirring or agitation of the culture medium for at least one hour, and preferably between 2 and 4 hours.

The spirulina, once harvested, can be dried to obtain a dehydrated or partially dehydrated spirulina composition.

Preferably, the drying is done at a temperature below 60° C., in order to avoid denaturing the active ingredients, particularly the vitamins, contained in the compositions.

The spirulina generally remain intact during these operations so that the desired active ingredients such as phycocyanin and SOD remain inside the cells. Thus, they are physically better protected.

Insofar as the combinations and compositions according to the invention have substantial phycocyanin contents, their assimilation by the body makes it possible to naturally stimulate erythrocyte synthesis. This ultimately produces better oxidation of the blood which, particularly in athletes, results in greater endurance in the event of physical effort and a greater capacity for recovery.

A combination or composition according to the invention, insofar as it also comprises substantial protein, polyunsaturated fatty acid and vitamin contents, can form a good dietary supplement. Since the products of the invention are natural products that can be obtained completely organically—without the use of pesticides—they are perfectly suitable for feeding to young children and even nursing infants.

Likewise, insofar as spirulina contain neither gluten nor lactose, it can be advantageous to use a combination or composition according to the invention in strict diets intended for nursing infants and for adults who are allergic to these ingredients, in order to provide them with a balanced, protein-rich diet.

Another subject of the invention consists in a medication comprising a combination or composition according to the invention.

A combination or composition according to the invention can also be used to produce a medication or a dietary supplement for increasing blood oxygenation capacities or for increasing capacities for recovery after effort.

A combination or composition according to the invention can also be used to produce a medication or a dietary supplement for offsetting deficiencies in the immune system of a mammal.

Since the combinations or compositions according to the invention are rich in antioxidant active ingredients, particularly SOD and phycocyanin, their use in cosmetics, and particularly in dermatology, is advantageous, particularly for formulating healing or anti-aging compositions.

### EXAMPLE

### 1/Phase for Seeding Spirulina Cultures of the LONAR and PARACAS Strain Types

A 1-m³ volume of “Spiru-plus No. 1” medium, whose composition is given in Table 1, is seeded with 1000 ml of respective pre-cultures of Arthrospira strains of the LONAR and PARACAS type. It is preferable in this case to use “long-lived cultures” in which the strains are cultivated separately, in order to be able to rebalance the culture with one of the two strain types, if necessary. Once seeded, the medium is stirred continuously during a first, so-called maturation phase. This maturation phase is carried out at 25° C. with an illumination phase of 12 hours per day of natural light. The pH of the medium is maintained between 10 and 1. The maturation phase lasts approximately 20 days.

TABLE 1
“SPIRU-PLUS” Formula No. I

| | | | |
|---|---|---|---|
| NaHCO₃ | Sodium Hydrogenocarbonate | 8-10 | g/l |
| NaCl | Sodium Chloride | 4-6 | g/l |
| K₂NO₃ | Potassium Nitrate | 1-4 | g/l |
| NH₄H₂PO₄ | Monoammonium Phosphate | 0.05-0.1 | g/l |
| K₂SO₄ | Dipotassium Sulfate | 1-2 | g/l |
| MgSO₄—7H₂O | Magnesium Sulfate | 0.1-1 | g/l |
| (NH₂)₂CO | Carbamide (=Urea) | 0.01-0.1 | g/l |
| CO₂ | Carbon Dioxide | 1-2 | g/l |

2/Physicochemical Stress Phase:

After 20 days of culture, a so-called “physicochemical stress” phase is applied by changing the composition of the culture medium. A new medium is used, the “Spiru-plus” No. 2 medium, whose composition is given in Table 2. The pH is lowered until it reaches a constant value between 8 and 10, at the time when the light is at its maximum variation during the day.

TABLE 2
“SPIRU-PLUS” FORMULA No. II

| | | |
|---|---|---|
| FeSO₄—7H₂O | Ferrous Sulfate | 0.01-0.1 g/l |
| E.D.T.A. | Ethylene Diamene Tetraacetic Acid | 0.02-0.1 g/l |
| CuSO₄—7H₂O | Copper Sulfate | 0.1-0.2 g/l |
| ZnSO₄—7H₂O | Zinc Sulfate | 0.1-0.2 g/l |
| MnCl2 —4H₂O | Manganese Chloride | 0.5-1.5 g/l |
| MoO₃ | Molybdenum Oxide | 0.01-0.5 g/l |

3/Cell Structuring and Rapid Maturation Phase

The culturing of LONAR and PARACAS type strains is carried out in a culture medium called “Spiru-Plus No. 3,” whose composition is given in Table 3. This medium enables more rapid cell growth and maturation of the spirulina strains. The culture is maintained under agitation at a pH between 8 and 11.

TABLE 3
“SPIRU-PLUS” FORMULA No. III

| | | |
|---|---|---|
| CACl₂ | Calcium Chloride | 0.02-0.08 g/l |
| NaNO₃ | Sodium Nitrate | 1-5 g/l |
| H₃BO₃ | Boric Acid | 2-5 g/l |
| Co(NO₃)₂—6H₂O | Cobalt Nitrate | 0.05-0.5 g/l |
| NiSO₄—7H₂O | Nickel Sulfate | 0.4-1.0 g/l |
| K₂Cr(SO₄)₄—24H₂O | Chromium Sulfate | 0.9-1.5 g/l |

It is noted that the culture media “Spiru-plus I, II and III,” under the conditions described above, are synthetic media. These media have the advantage of limiting the risk of contamination by microorganisms and parasites.

4/Rest Phase

In order for the spirulina to become richer in active ingredients, it is preferable to allow them one or more rest phases, during which the culture medium is not stirred.

6/Active Ingredient Content of the Mixed Cultures of Arthrospira sp. LONAR and Arthrospira sp. PARACAS (the Combination According to the Invention) and Comparison with the Product of Individual Cultures of the Same Strains.

The Arthrospira sp. LONAR and Arthrospira sp. PARACAS strains were cultivated according to the above-described protocol.

Once the cultures were harvested and filtered, the spirulina mixture was dehydrated in an oven maintained at a temperature below 60° C. The powder obtained, called “HTPA,” was finely ground before proceeding with the analysis presented in Table 4 below.

The Arthrospira sp. LONAR and Arthrospira sp. PARACAS spirulina strains were also cultivated separately following the same protocol. After harvesting and dehydration, the products derived from the two cultures were mixed, then analyzed. The results of the analyses appear on the right-hand side of the following table.

TABLE 4
SPIRULINA COMPOSITION
(mixed culture/separate Arthrospira sp. LONAR + PARACAS cultures)

| | mass/100 g | | | |
|---|---|---|---|---|
| | (dry weight) | | mass/100 g | |
| | mixed culture | | (dry weight) | |
| AMINO ACIDS | HTPA | | separate cultures | |
| Cysteine + Cystine | 0.56 | g | 0.51 | g |
| Total aspartic acid | 5.7 | g | 6.1 | g |
| Total threonine | 3 | g | 2.85 | g |
| Total serine | 3.28 | g | 3.04 | g |
| Total glutamic acid | 8.8 | g | 9.3 | g |
| Total proline | 2.23 | g | 2.28 | g |
| Total glycine | 3.04 | g | 2.93 | g |
| Total alanine | 4.7 | g | 4.5 | g |
| Total valine | 4 | g | 4 | g |
| Total methlonine | 1.36 | g | 1.26 | g |
| Total isoleucine | 3.6 | g | 3.5 | g |
| Total leucine | 5.3 | g | 5 | g |
| Total tyrocine | 3 | g | 2.8 | g |
| Total phenylalanine | 2.61 | g | 2.53 | g |
| Total lysine | 2.83 | g | 2.75 | g |
| Total histidine | 1.05 | g | 1.04 | g |
| Total arginine | 4 | g | 4.6 | g |
| Total tryptophan | 0.94 | g | 0.9 | g |
| PIGMENTS | mass/100 g | | mass/100 g | |
| Total phycocyanin | 16.85 | g | 12.10 | g |
| ENZYMES | activity | | activity | |
| Superoxide dismutase | 215,000 | IU | 160,000 | IU |

It may be seen from the results of these analyses that, for an equivalent amino acid profile, the phycocyanin and SOD content is significantly higher when the spirulina strains Arthrospira sp. LONAR and Arthrospira sp. PARACAS are cultivated simultaneously. The phycocyanin content in the HTPA spirulina represents 16.8% of the total dry weight, as opposed to 12.10% in the case of the separate cultures. The activity resulting from the presence of SOD (superoxide dismutase) is 215,000 IU per 100 g of dry composition, as opposed to 160,000 IU.

These results show that the simultaneous culturing of the Arthrospira sp. LONAR and Arthrospira sp. PARACAS strains makes it possible to synergistically obtain higher phycocyanin and SOD contents.

7/Active Ingredient Contents of a Combination According to the Invention and Comparison with a Spirulina of the Arthrospira pacifica Type

A complete profile of the mixed culture of Arthrospira sp. LONAR and Arthrospira sp. PARACAS spirulina was done. The results obtained appear in Table 5 below.

Table 6 lists the active ingredient contents of Arthrospira pacifica, which is reputed to be a spirulina of excellent quality. The values shown here are those provided by the producers of that spirulina.

It may be seen when comparing Tables 5 and 6 that the phycocyanin and linolenic and di-homo-gamma-linolenic fatty acid contents are significantly higher when Arthrospira sp. LONAR and Arthrospira sp. PARACAS are combined according to the method of the invention. The same is true for SOD activity, and to a lesser extent for other active ingredients such as, for example, vitamins A, D, E, K and B12.

TABLE 5
SPIRULINA COMPOSITION
(Combination of Arthrospira sp. LONAR and PARACAS strains)

| | % mass by dry weight | |
|---|---|---|
| PIGMENTS | | |
| (analysis by spectrophotometry) | | |
| Phycocyanin | 16.85 | |
| Chlorophyll | 1.2 | |
| Carotenoids | 0.4 | |
| Beta-carotene included in the above | 0.18 | |
| FATTY ACIDS | | |
| (analysis by CPG chromatography) | | |
| Palmitic acid | 2.37 | |
| Palmitoleic acid | 0.40 | |
| Stearic acid | 0.18 | |
| Oleic acid | 0.22 | |
| Essential fatty acids | | |
| Linoleic acid | 1.10 | |
| Gamma linoleic acid | 1.89 | |
| Di homo gamma linoleic acid | 0.25 | |
| VITAMINS | mass mg/10 g | |
| B1 Thiamine | 0.35 | |
| Vitamin B3 | 1.6 | |
| Vitamin B9 - folic acid | 0.1 | |
| Vitamin E - tocopherol | 1.2 | |
| Vitamin PP - Niacin | 1.6 | |
| Vitamin B5 - Pantothenic acid | 10 | |
| Beta carotine | 140 | |
| Vitamin C | 0 | |
| Vitamin B2 - Riboflavin | 0.40 | |
| Vitamin B6 | 0.8 | |
| Vitamin B12 - Cobalimin | 0.29 | |
| Vitamin K - Phylloquinone | 0.2 | |
| Vitamin B8/ = H Biotin | 5 | |
| Inositol | 6.4 | |
| | activity | |
| Vitamin A | 24,000 | IU |
| Vitamin D | 1,200 | IU |
| ENZYMES (per 100 g) | | |
| Superoxide dismutase (SOD) | 215,000 | IU |

| | mass | | mass |
|---|---|---|---|
| | mg/10 g | | mg/10 g |
| MINERALS | (dry weight) | MINERALS | (dry weight) |
| Ca | 100 | Cl | 0.1 |
| Cr | 0.35 | Na | 20 |
| Io | 0.0 | Se | 1.9 |
| Fe | 18 | Mn | 1 |
| K | 140 | Mb | 0.2 |
| Cu | 1.8 | Mg | 40 |
| Br | 9 | Zn | 0.3 |
| P | 80 | Ge | 0.9 |

TABLE 6
SPIRULINA COMPOSITION
(Arthrospira pacifica)

| Essential amino acids | % mass by dry weight |
|---|---|
| Isoleucine | 3.26 |
| Leucine | 4.89 |
| Lysine | 2.62 |
| Methionine | 1.33 |
| Phenylalanine | 2.61 |
| Threonine | 2.81 |
| Tryptophan | 0.85 |
| Valine | 3.74 |
| Non-essential amino acids | |
| Alanine | 4.66 |
| Arginine | 4.76 |
| Asparagic acid | 7.28 |
| Cysteine | 0.56 |
| Glutamic acid | 8.44 |
| Glycine | 3.19 |
| Histidine | 1.50 |
| Proline | 2.47 |
| Serine | 2.65 |
| Tyrosine | 2.38 |
| Fatty acid | |
| Gamma-linolenic acid | 1.0 |
| Essential linolenic acid | 1.10 |
| Di-homo-gamma-linolenic acid | 0.05 |
| Alpha-linolenic acid | 0.01 |
| Oleic acid | 0.02 |
| Palmitic acids | 2.0 |
| Oleic-palmitic acids | 0.19 |
| Steric acids | 0.01 |
| Pigments | |
| Chlorophyll A | 0.790 |
| Beta carotenes | 0.28 |
| Carotenoids | 0.54 |
| Phycocyanin | 11.1 |
| Enzymes | |
| Superoxide dismutase | 0.04 |

| | mass mg/kg of dry weight |
|---|---|
| Minerals | |
| Calcium (Ca) | 4,000 |
| Iron (Fe) | 1,060 |
| Potassium (K) | 15,200 |
| Magnesium (Mg) | 4,800 |
| Manganese (Mn) | 26 |
| Molybdenum (Mo) | 1.50 |
| Sodium (Na) | 7,300 |
| Phosphorus (P) | 10,400 |
| Selenium (Se) | 2.5 |
| Zinc (Zn) | 18 |
| Vitamins | |
| Beta carotenes (provitamin A) | 2,800 |
| Thiamine (B1) | 34 |
| Riboflavin (B2) | 33 |
| Niacin (B3) | 207 |
| Pantothenic acids (B5) | 4 |
| Pyridoxine (B6) | 4.4 |
| Cyanocobalamin (B12) | 1.1-2.2 |
| Delta-alpha tocopherol (E) | 15 |
| Biotin (H) | 0.4 |
| Folic acids | 0.3 |
| Inositol | 680 |

1-22. (canceled)

23. A spirulina combination comprising at least two distinct strain types from the genus Arthrospira having a phycocyanin content of between 13 and 30%.

24. The combination according to claim 23, wherein said strains also comprise SOD having an activity of between 60,000 and 300,000 IU per 100 dry g of said strains.

25. The combination according to claim 23, wherein the composition also comprises a di-homo-gamma-linolenic acid content of between 0.05 and 2%, (% by dry weight).

26. The combination according to claim 23, wherein said composition also comprises a gamma-linolenic acid content of between 0.5 and 5%, (% by dry weight).

27. The combination according to claim 23, wherein the weight ratio of the two strain types is 80:20, (by dry weight).

28. The spirulina combination according to claim 23, wherein the two strain types are Arthrospira sp. LONAR and Arthrospira sp. PARACAS.

29. A composition comprising a spirulina composition according to claim 23.

30. The composition according to claim 29, wherein the composition comprises (% by dry weight):
10-70% spirulina of the Arthrospira sp. LONAR type;
10-70% spirulina of the Arthrospira sp. PARACAS type;
0-30% spirulina of another type.

31. The composition according to claim 29, wherein the phycocyanin content, and if applicable the SOD content, is intracellular.

32. The composition according to claim 29, wherein the composition also comprises at least one active ingredient from the group consisting of: isoflavone, vitamin C, bamboo exudates and Pygeum africanum extracts.

33. A method for preparing a combination according to claim 23, comprising:
a) cultivating at least two types of spirulina strains in the same culture medium; and
b) the spirulina cultivated in a) are harvested.

34. A method for preparing a spirulina combination comprising:
i) culturing at least two types of spirulina strains in a culture medium whose pH is between 8 and 12; and
ii) applying physicochemical stress to the culture medium by varying the pH within a range of 1 to 3 points, during a luminosity peak.

35. The method according to claim 33, wherein the at least two types of spirulina strains cultivated are Arthrospira sp. LONAR and Arthrospira sp. PARACAS.

36. The method according to claim 33, including allowing the culture medium to rest prior to harvesting the spirulina.

37. The method according to claim 34 for obtaining a dehydrated or partially dehydrated spirulina composition comprising drying the spirulina compositions obtained at a temperature below 60° C.

38. A spirulina combination, prepared by the method according to claim 33.

39. A dietary supplement comprising a combination according to claim 23.

40. A medication comprising a combination according to claim 23.

41. A method for increasing blood oxygenation capacities in a mammal comprising administering to a mammal in need thereof an effective amount of a combination according to claim 23.

42. A method for increasing a mammal's capacity for recovery after effort comprising administering to a mammal in need thereof an effective amount of a combination according to claim 23.

43. A method for offsetting deficiencies in the immune system of a mammal comprising administering to a mammal in need thereof an effective amount of a combination according to claim 23.

44. A cosmetic composition comprising a combination according to claim 23 and a cosmetically acceptable carrier.
